(19) United States
(12) Patent Application Publication
(10) Pub. No.: US 2026/0133315 A1
(43) Pub. Date: May 14, 2026

(54) HIGH FREQUENCY ULTRASOUND SYSTEM

(71) Applicant: URSUS MEDICAL DESIGNS LLC, PITTSBURGH (CA)

(72) Inventors: Sergei Koptenko, Mississauga (CA); Thomas Hayes, Allison Park, PA (US)

(21) Appl. No.: 19/347,659

(22) Filed: Oct. 1, 2025

Related U.S. Application Data
(60) Provisional application No. 63/702,135, filed on Oct. 1, 2024.

(51) Int. Cl. G01S 15/89; A61B 8/00; G01S 7/52

(52) U.S. Cl. CPC G01S 15/8956; A61B 8/4488; A61B 8/461; G01S 7/52057

(57) ABSTRACT

An ultrasound system includes an ultrasonic array having at least one ultrasonic element, wherein the ultrasonic array configured for transmitting analog ultrasound signals in ultra-high frequency above 25 MHz; an analog sampling recorder receiving the ultrasound signals in ultra-high frequency above 25 MHz and outputting an analog signal with a reduced frequency, wherein the analog sampling recorder will reduce the analog signal frequency; and an ultrasound subsystem coupled to the analog sampling recorder wherein the reduced frequency analog signal from the analog sampling recorder forms an analog front-end channel of the ultrasound subsystem.

### RELATED APPLICATIONS

The present application claims the benefit of provisional patent application Ser. No. 63/702,135 filed Oct. 1, 2024, which application is incorporated herein by reference.

### BACKGROUND INFORMATION

### 1. Field of the Invention

The present invention relates to ultrasonic beamforming as a way to receive and focus ultrasound signals to produce an image. More specifically, the present invention relates to high frequency (>25 MHz) ultrasound beamforming systems and associated methods.

### 2. Background of the Invention

There are several areas in the electronics field in which analog memory devices are being used successfully, such as in digital storage oscilloscopes, in the physics field X-ray and charged-particle tracking applications.

Some early predecessors of this technology can be traced to digital oscilloscopes and waveform capturing devices based on Fast-In-Slow-Out (FISO) principle, such as one described in U.S. Pat. No. 4,271,488 titled “High-Speed Acquisition System Employing an Analog Memory Matrix” or in U.S. Pat. No. 4,833,445 titled “FISO Sampling System”. These patents are incorporated herein by reference and the latter patent depicts the fast, high resolution FISO system, while the former describes an acquisition system that uses an analog memory matrix built of sample-hold cells arranged in rows and columns to form an M×N matrix that may be implemented on a single integrated-circuit (IC) chip.

The idea of a matrix analog memory device on IC was further developed by Stewart Kleinfielder who produced a range of multichannel transient analog waveform digitizer chips used to capture data from detectors in neutrino physics experiments, as well as by other contributors (for example, see Kleinfelder, S. A., “A 4096 Cell Switched Capacitor Analog Waveform Storage Integrated Circuit”, IEEE Transactions on Nuclear Science, NS-37, No. 1, February 1990; and Kleinfelder, S. A., “Advanced Transient Waveform Digitizers,” SPIE Particle Astrophysics Instrumentation Proc., v. 4858, pp. 316-326, August 2002.) Additional prior art representing informative background can be found in U.S. Pat. No. 4,099,251 titled “Analog Accumulator Memory Device”; U.S. Pat. No. 5,722,412 titled “Hand Held Ultrasonic Diagnostic Instrument”; U.S. Pat. No. 6,126,602 titled “Phased Array Acoustic Systems with Intra-Group Processors”; U.S. Pat. No. 8,220,334 titled “Transducer Array Imaging System”; U.S. Pat. App. Pub. No. 2004-0015079A1 titled “Ultrasound Probe with Integrated Electronics”; U.S. Pat. App. Pub. titled No. 2008-0262351A1 “Microbeamforming Transducer Architecture”; U.S. Pat. App. Pub. No. 2010-0152587A1 titled “Systems and Methods for Operating a Two-Dimensional Transducer Array”; and U.S. Pat. App. Pub. No. 2011-0213251A1 titled “Configurable Microbeamformer Circuit for an Ultrasonic Diagnostic Imaging System.” These patents and publications are incorporated herein by reference. See also Haller, G. M.; Wooley, B. A., “A 700-MHz switched-capacitor analog waveform sampling circuit,” IEEE Journal of Solid-State Circuits, v.29 (4), pp. 500-508, April 1994 and Kai E. Thomenius, “Recent Trends in Beamformation in Medical Ultrasound”, IEEE Ultrasonics Symposium 2005.

In medical diagnostic ultrasound, there were a number of attempts to use analog memory for ultrasound signal beamforming, notably in U.S. Pat. Nos. 6,500,120 and 6,705,995, both of which are incorporated herein by reference.

URSUS Medical Designs LLC developed Analog Sampling Beamformer (ASB) method and system outlined in patents: U.S. Pat. No. 9,739,875 titled “Analog Store-Digital Read (ASDR) Ultrasound Beamformer Method and a System”, U.S. Pat. No. 10,627,510 titled “Ultrasound Beamforming System and Method Based on Analog Random-Access Memory Array”, and U.S. Pat. No. 11,154,276 titled “Ultrasound Beamforming System and Method with Reconfigurable Aperture.” The above identified patents and published patent applications are incorporated herein by reference.

The process of ultrasound imaging, such as in medical diagnostic, begins with sending specially constructed ultrasonic signals (pulses, waves, or wave packets) into the subject, e.g., tissues in medical diagnostics (or turbine blades for jet engine inspection, etc.) The pressure pulse propagates in depth while attenuating and scattering on the acoustic impedance interfaces (such as a boundary between different tissues) along the way. These scattered echoes are picked up by the receiving ultrasound array and from this data the tissue composition along the pulse propagation path is reconstructed as a single scan line. Then, the next pulse is sent into a different direction and the process of receiving scattered (or attenuated as in transmission tomography) ultrasound signals back to the sensor array, and the interpretation of the results is repeated until a required 2-D slice (B-mode frame) or a 3-D volume is assembled out of separate scan lines.

In order to increase the spatial and contrast (magnitude) resolution of a signal coming from the certain spatial location within the tissue, the ultrasound array needs to be focused on that location. Thus, in the course of pressure pulse propagation in the tissue, the receiving array needs to constantly shift its focus following the pulse current position. Therefore, one of the first steps in processing the raw data is called beamforming in which signals coming to different elements of the array are time-shifted before they are added to one another. As a rule, the beamforming applies to both transmit and receive signals.

FIG. 1 illustrates the common method used in forming ultrasound images, also known as digital beamforming. Generally, the ultrasound imaging device consists of an ultrasonic array 106 divided into a number of independent elements 107 or channels (typically to 64 or 256 elements in linear or curved 1D array). During the transmit stage of interrogation, the transmit beamformer (not shown on this figure) sends variably delayed electric pulses to the elements of the ultrasound array 106. The relative delays between the signals are constructed in such a way that ultrasonic pulses emitted by elements 107 of the array 106 would arrive to the predetermined spatial point 100 (focal point P) simultaneously, with their phases aligned to achieve a coherent summation of wavelets coming from all elements 107 of the array 106. This wave would scatter at point 100 (and all other steep changes in acoustic impedance within the interrogated body) and part of this spherical scattered wave would travel back to the elements 107 of the array 106. Each element 107 would convert pressure variations in the incoming wave into the voltage variation output 108. The portion of this scattered wave that reaches the surface of an array element 107 can be seen as a wavelet 102 that travels along the ray 104 that connects the scattering point 100 and the face of the element 107. Depending on the mutual position of the scattering point 100 and the specific element 107 of the array 106, the length of the path 104 would vary from the shortest one, equivalent to radius R0 105 to the longest one. The spatial difference ΔDi between the shortest path 105 and path from the point 100 to the i-element of the array 106 translates into the time delay Δti between the arrivals of signals 108. The task of the receive beamformer is to modify the time differences between the signals 108 from all elements 107 participating in beamforming and sum them in accordance with the directions of the beamforming algorithm. For example, such a beamforming algorithm may require removing the time delays Δt from all arrived signals and sum such processed signals (delay-sum algorithm), in effect focusing the array to the point P. It can be seen that the workings of transmit and receive beamformers are mutually reciprocal, thus, describing the works of the receive beamformer is also a description of the solutions for the transmit beamformer.

The ways received signals are processed define the type of beamformer. The common type of receive side digital beamformer used to process ultrasound signals is shown on FIG. 1. In the digital beamformer, voltage signals 108 from the elements of the array 106 are amplified by the voltage controlled amplifier (VCA) 110 to compensate the signal attenuation, then, the signal in each channel is digitized at a certain sampling rate by channel ADC 124 that outputs digitized signal to the memory or First-In-First-Out (FIFO) registers where signals are shifted in accordance with the beamforming algorithm (for example such that to remove arrival delay Δt), then such processed digital data 128 from each participating channel are summed by digital summator 130 and output data 122 are written to the memory for further processing. The advantages of digital beamformer, such as shown in FIG. 1, are its speed and precision which allows implementation of the dynamic beamforming and the possibility of realization of multiple beamforming strategies on the same data volume. What can be seen as disadvantage of this approach is the relative complexity of the hardware, manifesting in larger hardware size, higher cost, and higher power consumption as compared to other beamforming methods such as analog and analog sampling beamformers.

For the reasons of clarity, the beamforming schematic for the receive side digital beamformers shown on FIG. 1 was simplified by removing the multiplexing stage. However, in reality, as known to those of ordinary skill in the art, having the number of processing channels be equal to the number of arrays' elements is not always possible. The actual design could be limited by physical dimensions, cost, or power availability. Thus, the ultrasound array can have 64, 128, 256 or greater number of elements, but the beamformer would have typically 64 or 128 channels with an analog multiplexing circuitry 200 shown on FIG. 2 that would select elements of the array 106 into the current aperture connecting it to receive channel 109 and transmit channel 204.

A simplified block diagram of the ultrasound system 150 is shown in FIG. 2 that also shows the High Voltage Transmit-Receive switches 202 that connect or disconnect the receive side of the beamforming channel 109 from the element of array 107.

From the description of the beamforming process, it can be seen that the signal coming from the output of the array element 107 is processed independently from the signals coming from the other elements up to the output of the beamformer where all of the signals are combined. Thus, this text will refer to this signal path from element 107 to the input Low Noise Amplifier 206, Voltage-Controlled Amplifier 208 Low Pass Filter block 210, and Analog-to-Digital Converter 212 as a “signal path” or “beamforming channel” or simply as “channel” 109.

The remaining blocks on FIG. 2: Transmit Beamformer (BF) 214, Receive BF 218, BF Control Unit 216, Preprocessing Unit 220, Scan Conversion Unit 222, Ultrasound UI 224, and Display or a Computer System 160, schematically represent typical systems blocks as known to those of ordinary skill in the art.

Considering all the limitations discussed above, the design of ultrasound diagnostic systems for a standard clinical range of frequencies from 2 MHz to 25 MHz is pretty well established. However, new areas of research and diagnostics, such as ultrasound dermatology, small animal research, ultrasound histology, ultrasound biomicroscopy, NDT and many others require significant raise in frequency bandwidth, from 30 MHz potentially up into the gigahertz range (ultra-high ultrasound). Considering that realistic sampling rate of the receive channel ADC should be 5 to 10 times the highest frequency in the useful bandwidth, while maintain at least 12 to 14 bits resolution, the complexity and the cost of the hardware for 64 or 128 channels system becomes prohibitively high.

There remains a need in the art to reduce the cost of the hardware, size, and power requirements of high and ultra-high frequency ultrasound imaging and to utilize beamforming architecture to accomplish this goal.

### SUMMARY OF THE INVENTION

According to principle of the present invention, this high and ultra-high frequency ultrasound (UHF-US) beamforming architecture performs the task of signal conditioning using a High Frequency Wave Recorder 250 that employs an array of sample/hold cells to capture, store and process instantaneous samples of analog signals from the ultrasound array element 107 to record an analog waveform at the high sampling speed and play it back to the input of lower bandwidth ultrasound machine 150 at much lower sampling rate as schematically shown on FIG. 3A.

This architecture provides significant reduction in hardware complexity, power consumption and the size of the diagnostic ultrasound imaging system.

Main blocks of such UHF-US system (shown in FIG. 3B) are:
a) ultrasound probe array 106 comprising of at least one element 107 that can form 1D or 2D array of arbitrary form or shape or present a single element array shaped as directed by application's need. Some or all elements of array 107 could work as transmitting and receiving elements, while other elements 107 could be only transmitting or only receiving elements of the array. Transducer array 106 is capable of transmitting and receiving ultrasound signals in ultra-high frequency above normal clinical ultrasound range of 25 MHz, preferably greater than or equal to 30 MHz, possibly greater than or equal to 50 MHz, possibly greater than or equal to 100 MHz, possibly greater than or equal to 150 MHz, possibly greater than or equal to 200 MHz, possibly greater than or equal to 250 MHz, possibly less than or equal to 300 MHz, with a preferred operation within the 30 MHz to 300 MHz range.
b) High frequency analog buffer preamplifiers 208.
c) Analog Sampling Recorder (ASR) block 250 with Calibration block 254 and Synchronization block 252 these functions will be explained in detail in the following sections.
d) Ultrasound system 150 that could be a standard diagnostic ultrasound machine.
e) Display unit 160, a monitor or a smartphone, tablet, laptop, desktop, or any other computer system capable of receiving, transmitting, processing and displaying ultrasound images and cineloops.

One aspect of the present invention provides an ultrasound system comprising an ultrasonic array having at least one ultrasonic element, wherein the ultrasonic array configured for transmitting analog ultrasound signals in ultra-high frequency above 25 MHz; an analog sampling recorder receiving the ultrasound signals in ultra-high frequency above 25 MHz and outputting an analog signal with a reduced frequency, wherein the analog sampling recorder will reduce the analog signal frequency; and an ultrasound subsystem coupled to the analog sampling recorder wherein the reduced frequency analog signal from the analog sampling recorder forms an analog front-end channel of the ultrasound subsystem.

One aspect of the invention provides a signal sampling recorder receiving the ultrasound signals in ultra-high frequency above 25 MHz and outputting an analog signal with a reduced sampling frequency.

One aspect of the invention provides An ultrasound method comprising the steps of: defining a pulse shape; sending properly timed voltage pulses through HV multiplexors into the elements of a transducer array that convert voltage signals into the pressure pulses propagating into the target media; switching to receive mode whereby the elements of the transducer array receive portions of pressure waves from the target media; processing the received signals from the elements of the transducer array into inputs of an analog sampling recorder; storing the inputs of an analog sampling recorder for each element as a sequence of voltage samples at sampling rate f_{HS} in a memory buffer; outputting voltage samples of the memory buffer at sampling rate f_{LS} to the input of a channel A/D converter, wherein f_{HS}/f_{LS} is at least 5; and signal processing an output of the A/D converter to obtain an ultrasound image on a display.

The advantages of the present invention will be clarified in the brief description of the preferred embodiment taken together with the drawings in which like reference numerals represent like elements throughout.

### BRIEF DESCRIPTION OF THE DRAWINGS

FIG. 1 is a schematic representation of a prior art digital beamformer.

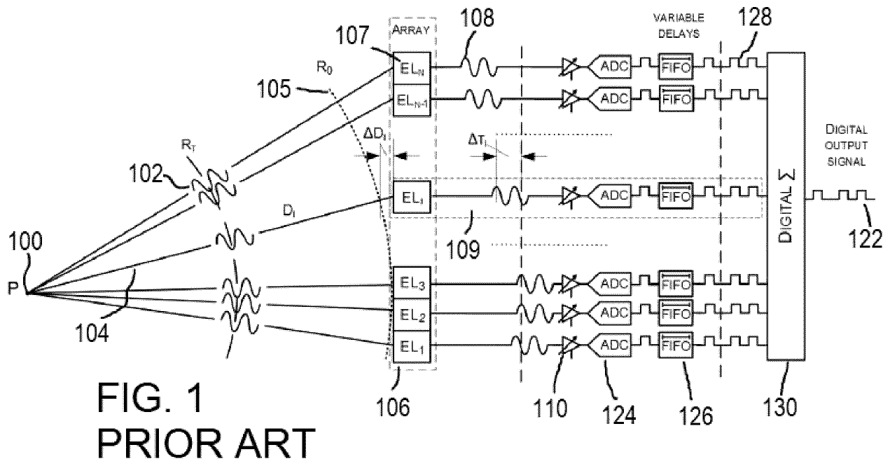

FIG. 2 is a schematic representation of a prior art digital beamformer-based ultrasound system.

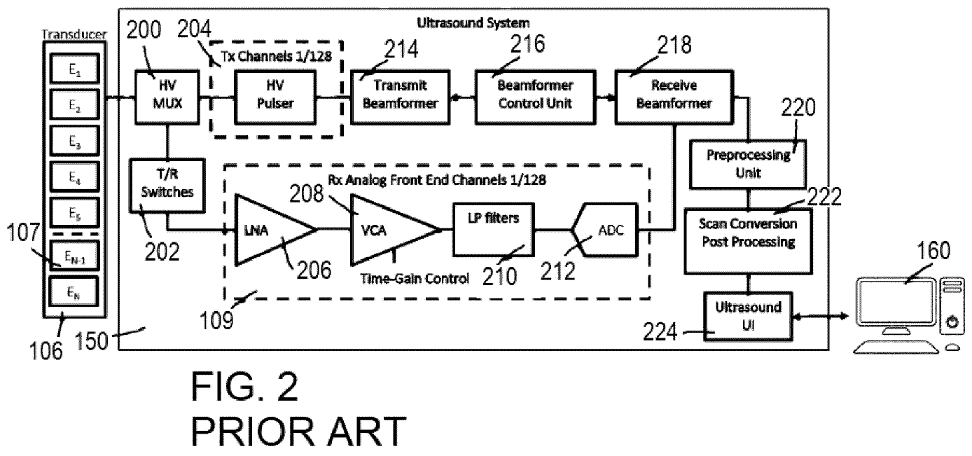

FIG. 3A-B are schematic representations of main blocks of ultra-high frequency ultrasound system (UHF-US).

FIGS. 4A-B are schematic representations of representative Analog Sampling Recorder (ASR) block for use in the UHF-US of the present invention.

FIG. 5 is a schematic representation of one embodiment of ultra-high frequency ultrasound system (UHF-US) of the present invention.

### DESCRIPTION OF THE PREFERRED EMBODIMENTS

The present invention relates to ultrasound diagnostic systems, such as used in medical diagnostic systems for medical human and animal applications. Some aspects of the present invention are understood in connection with inventor's prior work in U.S. Pat. No. 9,739,875 titled “Analog Store-Digital Read (ASDR) Ultrasound Beamformer Method and a System”, U.S. Pat. No. 10,627,510 titled “Ultrasound Beamforming System and Method Based on Analog Random-Access Memory Array”, and U.S. Pat. No. 11,154,276 titled “Ultrasound Beamforming System and Method with Reconfigurable Aperture” which are incorporated herein by reference. The system and method of the present invention is also applicable to non-destructive testing/evaluation commonly used to find flaws in materials and to measure the thickness of objects (e.g., ultrasound microscopy, semiconductor wafers and dies quality control, material testing, structural and manufacturing testing).

The system and method of the present invention is also applicable to biomicroscopy and ultrasound histology applications, and generally any ultrasound imaging (or image-like) applications requiring ultra-high frequency beamforming for transmission and/or receiving. The present invention is directed, in particular, the way signals coming from the elements of an ultrasonic array (receive beamformer) and going to the elements of the same array (transmit beamformer) are treated. The invention describes an improved beamformer system that provides better image quality combined with significant reduction in systems' size, power consumption and production cost as compared to current systems. Thus, even though the main area of application of this invention is in medical ultrasound, this beamforming architecture and the hardware and software built upon its principles can be used in other areas such as non-destructive testing, sonar, radar, terahertz, infrared, optical imaging systems, just to name a few examples.

The general idea of the new design is to create an analog sampling recorder that would take a high frequency analog signal as an input from an element 107 of ultrasound array 106, sample it at high sampling rate f_{HS} (e.g., 1 GigaSample per second), and then replay this record at much lower rate f_{LS} (e.g., 33 MegaSample per second) to the input of the standard ultrasound system, effectively reducing the analog signal frequency

$$\frac{{f}_{HS}}{{f}_{LS}} = 30$$

times in this example. The ultrasound system 150 processes signals and output an ultrasound image for original signals with 150 MHz-350 MHz bandwidth as if it would be a signal with 5 MHz- to 15 MHz bandwidth. The analog sampling recorder 250, 256 according to the invention will reduce the analog signal frequency such that f_{HS}>f_{LS}, preferably by at least five times, more preferably at least ten times, with twenty or thirty or more being possible.

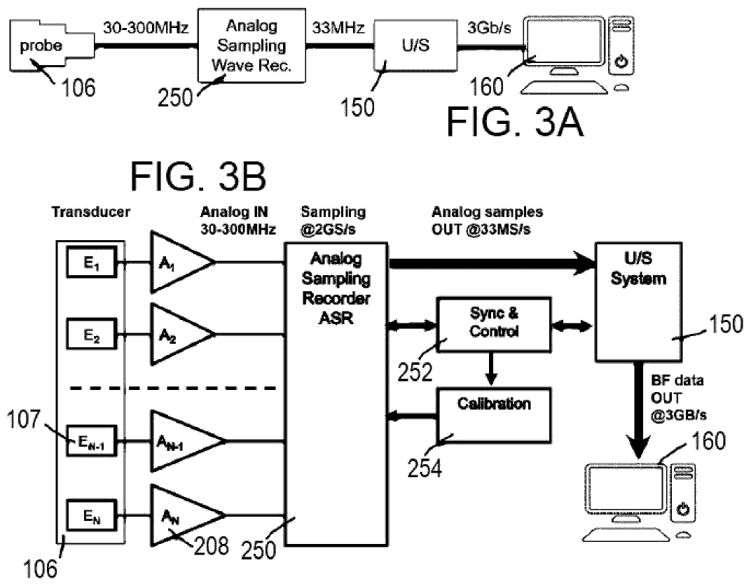

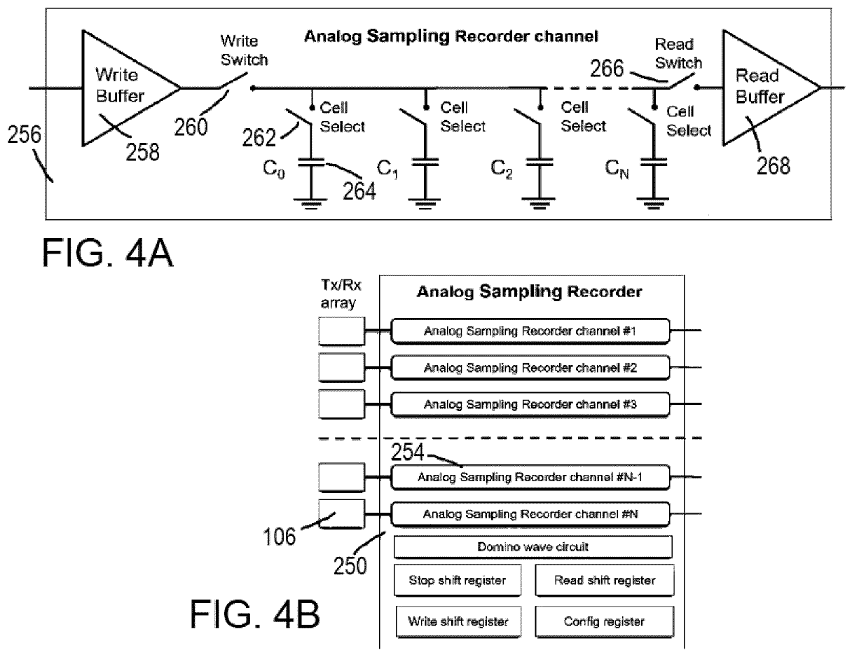

FIGS. 4A and 4B depict a schematic outline of an Analog Sampling Recorder 250 built with sequential write-read principle. Consider one ASR channel 254 depicted on FIG. 4A. The analog signal from the element 107 of the array 106 goes through an amplifier 208 (refer to FIG. 3B) to compensate the signal attenuation in the media, then, through Write Buffer 258 and through the closed Write Switch 260 is sequentially written at a certain sampling rate f_{HS} into an array of Sample-Hold Cells 262 controlled by Cell-Select switches 262 as a sequence of voltage levels.

During the read stage, write switch 260 is open and read switch 266 is closed. Cell-select switches 262 sequentially connect the storage capacitors 264 C₀, C₁ . . . C_{N} to the input of Read Buffer 268 at sampling rate f_{LS} providing voltage levels to the input of the analog front-end channel 109 of ultrasound system 150. More accurately in this invention the analog front-end channel 109 of ultrasound system 150 is forming an ultrasound subsystem 150 of the ultrasound system of the invention with the ultrasound system 150 having conventional elements from the analog front end channel 109.

Sampling rates f_{HS} and f_{LS} may be fixed, variable, changing from scan line to scan line, depth dependent or governed by some other relationship. Apart the analog channels 254 for every transducer element, ASR 250 also comprises of supporting circuitry (FIG. 4B)—such as read and write registers, domino wave circuit that facilitates the function of sequential cell select for write and read operations, registers that store the configuration information and other supporting circuitry. The architecture of sampling recorder shown on FIGS. 4A and 4B is given only as a simplified example of a typical system blocks as known to those of ordinary skill in the art.

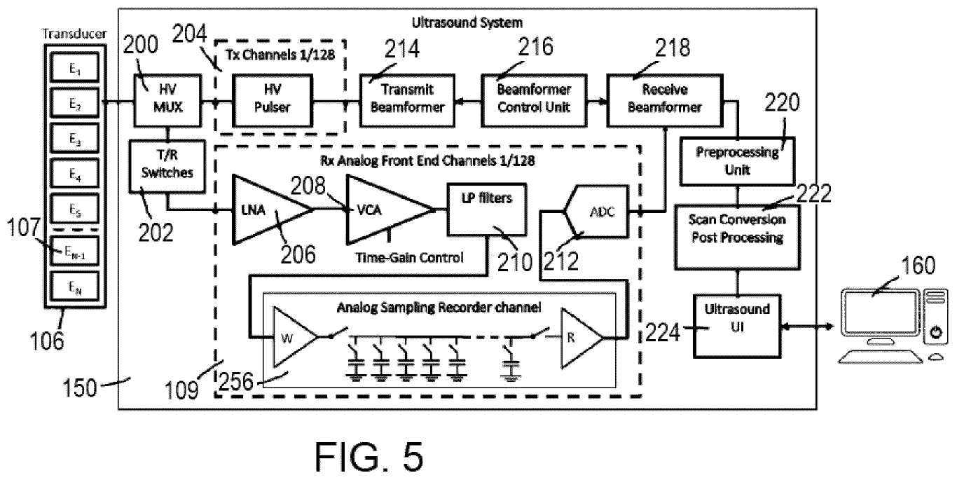

FIG. 5 schematically presents an example of an embodiment of the ultra-high frequency ultrasound system UHF-US that incorporates the ASR into a standard beamforming architecture.

The combination of Sample-Hold cells with read, write and cell-select switches can also be called an analog memory buffer or ASR channel 256. The design of sample-hold cells is well known and comprises prior art. Here a sample-hold cells design is used based on the storage capacitor as an example of the design; however, any device that can store an analog quantity can be used for building such a cell. Switches 260, 262, 264 can be made based on transistors, MEMs, or other technology enabling analog switching and multiplexing.

To calculate the required depth of the analog memory buffer of the ASR consider a standard imaging question—how many samples are required to capture a scan line at the given frequency? Using 3 MHz as a high-end of the frequency bandwidth, the sampling rate of 15 MHz (×5 times the frequency) and 25 cm as a maximum penetration depth we know from experience (also reflecting an empirical 500λ path criteria), we get the answer as ˜5,000 samples per scan line, from 2×250 mm divided by 0.1 mm (15 MHz wavelength). At the sampling rate of 30 MHz or ×10 times the frequency we would need 10,000 sample points.

In the preferred embodiment, with reference to FIG. 5, the ultrasonic system is constructed according to the principles of the present invention shown in block form. Referring to the FIG. 5, the transmit phase of beamformer operations begins with defining a pulse shape and transmit delay for every channel of the Transmit Beamformer 216, writing that information into the channel's High Voltage Pulser 204, sending properly timed voltage pulses through HV multiplexors 200 into the elements 107 of the transducer array 106 that convert voltage signals into the pressure pulses or wavelets that began propagating into the target media (tissue). At this point, ultrasound system switches to receive mode, connecting element 107 to LNA 206 via T/R switch 202. Portions of pressure waves scattered off the acoustic impedance inhomogeneities and impedance interfaces make way back to the transducer arrays elements 107, and after being amplified and filtered by the analog front-end section 206, 208 and 210 arrive to the input of the channel of analog sampling recorder 256 associated with the transducer element 107 selected by HV MUX 200. ASR 256 stores incoming continuous voltage signal as a sequence of voltage samples at sampling rate f_{HS} starting with the first cell 264 in memory buffer until it reaches the last memory cell. Then ASR switches to read-out mode and post voltage samples starting with the first cell at sampling rate f_{LS} to the input of the channels A/D converter 212. The remainder of the signal processing from the output of the ADC 212 to the final image display 160 via beamformer 218, preprocessing unit 220 and scan conversion post processing 222 and ultrasound UI 224 is well known to those of ordinary skill in the art. After read-out stage, the ultrasound system began forming another transmit event as it was described above, until it directed to stop the imaging operations by the control software.

In the preferred embodiment the depth of the analog memory buffer is sufficient to store enough samples to reconstruct a whole scan line as it was calculated above.

In other embodiments the depth of the analog memory buffer could be smaller than a full scan line. In this case, a full scan line depth could be procured by sequentially acquiring segments or stretches of scan line and then stitching these segments into the full line at the pre-processing stage. As an example, sampling 30 MHz signal at 150 MHz sampling rate we will get a full scan line of 5,000 samples to the depth of 25 mm. If our analog buffer 256 has 2000 sampling cells, we will need transmit-receive operations to obtain three scan line stretches 0 mm to 10 mm, 7.5 mm to 17.5 mm and 15 mm to 25 mm in depth to recover the full 0 mm to 25 mm scan line. Overlaps are needed to beamform channel data with proper delays with samples coming from the same acquisition.

In other embodiments, the ASR 256 is organized as a two-stage buffer where first buffer is a short-length analog memory operating at sampling rate f_{HS} connected to the secondary full-length analog memory buffer that writes at an intermediate sampling rate f_{HS}<f_{LS} and outputs data to ADC 212 at f_{LS} sampling rate.

In other embodiments, the analog sampling memory buffers 256 for all channels are organized as a single large analog memory array allowing to trade the length of the record for a number of connected channels.

In other embodiments analog sampling memory buffer 256 is designed such that the channel's analog memory buffer is split into separate memory blocks to reduce the parasitic capacitance.

In other embodiments analog sampling memory buffer 256 is designed such that it allows simultaneous read and write operations.

In other embodiments analog sampling memory buffer 256 is designed such that it allows simultaneous read and write operations, such that read address is updated in such a way to select the proper sample from the channel's buffer (proper delay) for the summation of all selected channels outputs in a beamforming instance. The read address update can be done either via “address slipping” or “temporarily stalls” the delay as described in the U.S. Pat. No. 6,500,120 or via arbitrary read address as described in U.S. Pat. Nos. 9,739,875, 10,627,510, and 11,154,276.

In other embodiments the ASR 256 together with associated electronics is put into the handle of the probe, such that it is “seen” by the ultrasound machine at the cable input connector as a standard clinical ultrasound probe with the regular ultrasound bandwidth.

In other embodiments channel ASR 256 consists of two parallel buffers that store I and Q parts of the incoming quadrature signal

In other embodiments channels ASR 256 can be set to oversampling the incoming signal in order to improve signal-to-noise ratio via samples averaging.

In other embodiments the probe array could be either 1D, 2D, row-column 2D array or sparse array.

In other embodiments the array 106 is split into the plurality of receiving elements array and plurality of transmit elements. The T/R switches 202 and HV MUX 200 are not needed in such a design. The array 106 has elements 107 permanently connected (hard wired) to either output of the transmit pulser 204 or to input of LNA 206 on receive side. The distribution of elements 107 between transmit and receive side within the array can be any, as directed by someone of ordinary skill in the art.

In other embodiments the array 106 is split into the plurality of receiving elements array and plurality of transmit elements where transmit elements are designed as a single elements' arrays, shaped for spatial focusing if desired. Such design allows plane wave mode of operation as well as continuous Doppler, elastography or contrast imaging modes of operation.

In other embodiments separate transmit arrays could be designed as one single element transmit array or a number of transmit elements. Such single transmit elements could be variably shaped for spatial focusing, have different directivity diagrams, work at different frequencies than the receiving array or other Tx arrays, have a single transmit pulse generator or each have its own pulse generator.

In other embodiments the probe array 106 could be placed on a surgical instrument or attachment for instantaneous assessment of tissue characterization or typing (benign vs malignant) during the surgery or procedure

In other embodiments the probe array 106 could be placed on intravenous catheter or biopsy needle for instantaneous assessment of tissue characterization or typing (benign vs malignant) during the investigation or procedure

In other embodiments the ultrasound system 150 could be used for ultrasound-based tissue histology for instantaneous assessment of tissue characterization or typing (benign vs malignant) during the surgery or procedure.

The scope of the present invention is defined by the appended claims and equivalents thereto.

1-19. (canceled)

20. An ultrasonic array having at least one ultrasonic element, wherein the ultrasonic array configured for transmitting analog ultrasound signals in ultra-high frequency above 25 MHz;
An analog sampling recorder receiving the ultrasound signals in ultra-high frequency above 25 MHz and outputting an analog signal with a reduced frequency, wherein the analog sampling recorder will reduce the analog signal frequency; and
An ultrasound subsystem coupled to the analog sampling recorder wherein the reduced frequency analog signal from the analog sampling recorder forms an analog front-end channel of the ultrasound subsystem.

21. The ultrasound system according to claim 20, wherein the analog ultrasound signals in ultra-high frequency above 25 MHz from the element of the array goes through an amplifier, then, through a Write Buffer.

22. The ultrasound system according to claim 20, wherein the analog sampling recorder is configured to receive the ultrasound signals in ultra-high frequency above 25 MHz and sample it at a high sampling rate f_{HS} and then replay this record at a lower rate f_{LS} to the analog front-end channel of the ultrasound subsystem, wherein f_{HS}>f_{LS} thereby effectively reducing the analog signal frequency.

23. The ultrasound system according to claim 22, wherein the f_{HS}/f_{LS} is at least 10, and further including a display unit coupled to the ultrasound subsystem.

24. The ultrasound system according to claim 20, wherein the analog ultrasound signals in ultra-high frequency above 25 MHz from the element of the array goes through an amplifier, then, through a Write Buffer and through a closed Write Switch and is sequentially written at a sampling rate f_{HS} into an array of Sample-Hold Cells controlled by Cell-Select switches as a sequence of voltage levels.

25. The ultrasound system according to claim 24, wherein within a read stage, the write switch is open and a read switch is closed and Cell-select switches sequentially connect storage capacitors to an input of a Read Buffer of the analog front-end channel at sampling rate f_{LS} providing voltage levels to the input of the analog front-end channel of ultrasound subsystem.

26. The ultrasound system according to claim 25, wherein at least one of the sampling rates the f_{HS} and f_{LS} are continuously variable with at least one of time or depth or area of interest while preserving f_{HS}>f_{LS}.

27. The ultrasound system according to claim 26, wherein at least one of the sampling rates the f_{HS} and f_{LS} are one of step wise or continuously variable.

28. The ultrasound system according to claim 20, wherein the ultrasonic array is configured for transmitting analog ultrasound signals in ultra-high frequency above 250 MHz.

29. The ultrasound system according to claim 20, wherein the ultrasonic array is configured for transmitting analog ultrasound signals in ultra-high frequency between 30 MHz and 300 MHz and further including a display unit coupled to the ultrasound subsystem.

30. An ultrasound method comprising the steps of:
defining a pulse shape;
sending properly timed voltage pulses through HV multiplexors into the elements of a transducer array that convert voltage signals into the pressure pulses propagating into the target media;
switching to receive mode whereby the elements of the transducer array receive portions of pressure waves from the target media;
processing the received signals from the elements of the transducer array into inputs of an analog sampling recorder;
storing the inputs of an analog sampling recorder for each element as a sequence of voltage samples at sampling rate f_{HS} in a memory buffer;
outputting voltage samples of the memory buffer at sampling rate f_{LS} to the input of a channel A/D converter, wherein f_{HS}/f_{LS} is at least 5; and
signal processing an output of the A/D converter to obtain an ultrasound image on a display.

31. The ultrasound method according to claim 30 further including the steps of transmitting a delay for every channel of a Transmit Beamformer; and writing delay information into each channel's High Voltage Pulser following defining a pulse shape.

32. The ultrasound method according to claim 30, wherein the depth of the analog memory buffer is sufficient to store enough samples to reconstruct a part of scan line and the whole scan line record could be obtained by stitching sequential partial scan line records.

33. The ultrasound method according to claim 30, wherein the analog sampling recorder organized as a two-stage buffer where first buffer is a short-length analog memory operating at sampling rate f_{HS} connected to the secondary full-length analog memory buffer that outputs data to the channel A/D converter at the f_{LS} sampling rate.

34. The ultrasound method according to claim 30, wherein the analog sampling recorder for all channels are organized as a single large analog memory array allowing to trade the length of the record for a number of connected channels.

35. The ultrasound method according to claim 30, wherein the analog sampling recorder is designed such that the channel's analog memory buffer is split into separate memory blocks to reduce the parasitic capacitance.

36. The ultrasound method according to claim 30, wherein the analog sampling recorder is designed such that it allows simultaneous read and write operations.

37. A signal sampling recorder receiving the ultrasound signals in ultra-high frequency above 25 MHz and outputting an analog signal with a reduced sampling frequency.

38. The signal sampling recorder according to claim 37, wherein the sampling recorder is configured to receive the ultrasound signals in ultra-high frequency above 25 MHz and sample it at a high sampling rate f_{HS} and then replay this record at a lower rate f_{LS} to a front-end channel of the ultrasound subsystem, wherein f_{HS}>f_{LS} effectively reducing the analog signal frequency.

39. The signal sampling recorder according to claim 37, wherein the sampling recorder is configured to inputs as a sequence of voltage samples at sampling rate f_{HS} in a memory buffer and output voltage samples of the memory buffer at sampling rate f_{LS}, wherein f_{HS}>f_{LS}.
